(19) United States
(12) Patent Application Publication
(10) Pub. No.: US 2026/0061170 A1
(43) Pub. Date: Mar. 5, 2026

(54) PROXIMAL EMBOLIC PROTECTION DEVICE FOR CAROTID STENTING

(71) Applicant: Boston Scientific Scimed, Inc., Maple Grove, MN (US)

(72) Inventors: Aiden Flanagan, Galway (IE); Cass Alexander Hanson, St. Paul, MN (US); Michael Hughes, Galway (IE); Martin Lawrence Fawdry, Galway (IE)

(73) Assignee: Boston Scientific Scimed, Inc., Maple Grove, MN (US)

(21) Appl. No.: 18/818,959

(22) Filed: Aug. 29, 2024

Related U.S. Application Data
(60) Provisional application No. 63/536,244, filed on Sep. 1, 2023.

(51) Int. Cl. A61M 25/10; A61F 2/954; A61F 2/966; A61M 25/00

(52) U.S. Cl. CPC A61M 25/1011; A61F 2/954; A61F 2/966; A61M 25/0023; A61M 2025/0004; A61M 2025/1052; A61M 2025/1097

(57) ABSTRACT

A catheter for carotid artery stenting includes a proximal catheter segment adapted to terminate with its distal end within a common carotid artery (CCA) and a distal catheter segment adapted to terminate within an external carotid artery (ECA). A working lumen extends distally through the proximal catheter segment. A CCA occlusion balloon is secured within the distal region of the proximal catheter segment and a ECA occlusion balloon is secured within the distal region of the distal catheter segment. A first shunt port is disposed within the proximal catheter segment proximal of the CCA occlusion balloon and a second shunt port is disposed within the distal catheter segment distal of the ECA occlusion balloon. A shunt lumen extending between the first shunt port and the second shunt port allows antegrade blood flow from proximal of the CCA occlusion balloon to distal of the ECA occlusion balloon.

### CROSS-REFERENCE TO RELATED APPLICATIONS

This application claims the benefit of priority under 35 U.S.C. § 119 of U.S. Provisional Application No. 63/536,244, filed Sep. 1, 2023, the entire disclosure of which is hereby incorporated by reference.

### TECHNICAL FIELD

The disclosure is directed to performing stenting within the carotid artery. More particularly, the disclosure is directed to providing embolic protection such as proximal embolic protection during carotid stenting.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to several alternative designs, materials and methods of manufacturing medical device structures and assemblies, and the use thereof. An example may be found in a reverse flow catheter for performing carotid artery stenting. The reverse flow catheter includes a proximal catheter segment extending from a proximal region to a distal region including a distal end, the proximal catheter segment adapted to terminate with its distal end within a common carotid artery (CCA). A distal catheter segment extends from a proximal end to a region including a distal end, the distal catheter segment extending distally from the distal end of the proximal catheter segment, the distal catheter segment adapted to terminate within an external carotid artery (ECA). A working lumen extends through the proximal catheter segment and terminating at the distal end of the proximal catheter segment. A CCA occlusion balloon is secured within the distal region of the proximal catheter segment and an ECA occlusion balloon is secured within the distal region of the distal catheter segment. A first shunt port is disposed within the proximal catheter segment proximal of the CCA occlusion balloon and second shunt port is disposed within the distal catheter segment distal of the ECA occlusion balloon. A shunt lumen extends between the first shunt port and the second shunt port in order to allow antegrade blood flow from proximal of the CCA occlusion balloon to distal of the ECA occlusion balloon.

Alternatively or additionally, the shunt lumen may extend through part of the proximal catheter segment and through the distal catheter extension.

Alternatively or additionally, the shunt lumen may include an ovoid-shaped shunt lumen.

Alternatively or additionally, the reverse flow catheter may further include an inflation lumen extending to and fluidly coupled with the CCA occlusion balloon.

Alternatively or additionally, the inflation lumen may also extend to and may be fluidly coupled with the ECA occlusion balloon.

Alternatively or additionally, the working lumen may be adapted to accommodate retrograde blood flow therethrough.

Alternatively or additionally, the catheter body may have an 8 French outer diameter.

Alternatively or additionally, the working lumen may have a 6 French inner diameter.

Alternatively or additionally, the working lumen may be adapted to accommodate a stent delivery catheter extended through the working lumen and into the interior carotid artery (ICA).

Another example may be found in a catheter. The catheter includes a proximal catheter segment extending from a proximal region to a distal region including a distal end, the proximal catheter segment adapted to terminate with its distal end within a common carotid artery (CCA), the proximal catheter segment having an 8 French outer diameter. A distal catheter segment extends from a proximal end to a region including a distal end, the distal catheter segment extending distally from the distal end of the catheter body, the distal catheter segment adapted to terminate within an external carotid artery (ECA). A working lumen extends through the proximal catheter segment and terminates at the distal end of the proximal catheter segment, the working lumen having a 6 French inner diameter. A CCA occlusion element is secured within the distal region of the catheter body and an ECA occlusion element is secured within the distal region of the distal catheter segment. A blood shunt path extends from a position proximal of the CCA occlusion element to a position distal of the ECA occlusion element in order to allow antegrade blood flow from proximal of the inflatable CCA occlusion balloon to distal of the inflatable ECA occlusion balloon.

Alternatively or additionally, the catheter may further include a proximal blood shunt port fluidly coupled with a proximal end of the blood shunt path and a distal blood shunt port fluidly coupled with a distal end of the blood shunt path.

Alternatively or additionally, the working lumen may be adapted to accommodate retrograde blood flow through the working lumen simultaneously with a stent delivery catheter being advanced through the working lumen.

Another example may be found in a reverse flow medical device combination for performing carotid artery stenting. The reverse flow medical device combination includes a first catheter and a second catheter. The first catheter includes a catheter body extending from a proximal region to a distal region including a distal end, the catheter body adapted to terminate with its distal end within a common carotid artery (CCA). A working lumen extends through the catheter body and terminating at the distal end of the catheter body. A first inflatable balloon is secured within the distal region of the catheter body of the first catheter and adapted when inflated to occlude the CCA. The second catheter includes a catheter body extending from a proximal region to a distal region including a distal end, the catheter body adapted to terminate with its distal end within an internal carotid artery (ICA), and an expandable stent disposed over the catheter body. A second inflatable balloon is secured within the distal region of the catheter body of the second catheter. A blood lumen extends through the catheter body to a position distal of the second inflatable balloon in order to allow antegrade blood flow from a position proximal of the first inflatable balloon to a position distal of the second inflatable balloon.

Alternatively or additionally, the second catheter may further include a stent deployment sheath that is adapted to be withdrawn proximally in order to deploy the stent.

Alternatively or additionally, the second catheter may further include a post-stenting dilation balloon.

Alternatively or additionally, the second catheter may further include one or more flushing ports disposed proximal of the second inflatable balloon.

Alternatively or additionally, the second catheter may be adapted to extend coaxially with the first catheter.

Alternatively or additionally, the second catheter may be adapted to extend within the working lumen of the first catheter.

Alternatively or additionally, the second catheter may be adapted to extend next to the first catheter.

Alternatively or additionally, the first inflatable balloon may include a lumen extending therethrough adapted to accommodate the first catheter within the lumen.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view of a portion of a human head and neck, illustrating some of the vasculature within the neck;

FIG. 2 is a schematic view of a portion of a human anatomy, including an illustrative flow path formed between an illustrative arterial sheath extending from the femoral artery to the carotid artery and a venous sheath extending from the femoral vein;

FIG. 3 is a schematic view of an illustrative reverse flow catheter;

FIG. 4 is a cross-sectional view taken along the line 4-4 of FIG. 3;

FIGS. 5 through 7 are schematic views providing an example of using the illustrative reverse flow catheter of FIG. 3;

FIG. 8 is a schematic view showing an alternate feature of the illustrative reverse flow catheter of FIG. 1;

FIG. 9 is a schematic view of an illustrative catheter;

FIGS. 10 through 16 are schematic views showing an example of using the illustrative catheter of FIG. 9; and

FIG. 17 is a schematic view showing an alternate feature of the illustrative catheter of FIG. 9.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or”unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A variety of arterial diseases are known. Carotid Artery Disease (CAD) is an example of an arterial disease in which plaque lesions may develop within a patient's carotid artery. Because of the position of the carotid artery, and because the carotid artery normally carries oxygenated blood from the heart towards the brain, it will be appreciated that performing endovascular catheter procedures such as but not limited to carotid artery stenting within the carotid artery may cause particles dislodged from the lesion or lesions to flow upwards into the brain during the endovascular catheter procedures. Foreign material entering the brain may have deleterious effects on a patient. While distal protection devices may be used to help capture dislodged particles, it will be appreciated that such distal protection devices have to cross the lesion in order to reach a position distal of the lesion. The act of advancing and positioning a distal protection device may in itself dislodge particles from the lesion.

Proximal protection devices do not have to be advanced across the lesion. In some instances, a proximal protection device such as an arterial sheath may be advanced through a patient's arterial system to a point within the carotid artery. As an example, the proximal protection device may enter the arterial system via the femoral artery, although other access points are contemplated. In some instances, the proximal protection device may reach a point within the common carotid artery, which is proximal of where the common carotid artery bifurcates into the external carotid artery and the internal carotid artery. Inflating an inflatable balloon at the distal end of the arterial sheath can occlude anterograde blood flow through the common carotid artery. By fluidly coupling a proximal end of the arterial sheath with the venous sheath, and because of the pressure differences between the arterial system and the venous system, retrograde blood flow may be created. As a result, any particles or other debris that may be dislodged from the lesion during a process of advancing the arterial sheath through the vasculature as well as during any interventional process such as stenting, will flow backwards through the arterial sheath and through the venous sheath and into the venous system.

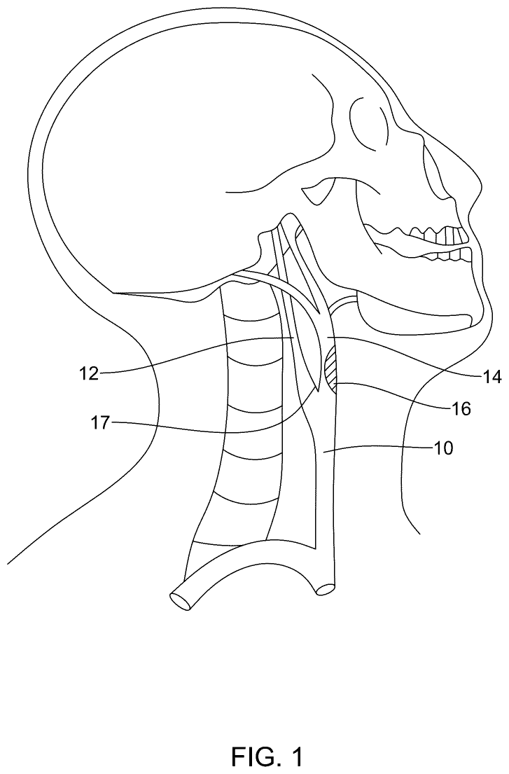

FIG. 1 is a partial cut-away view of the human head and neck, showing some of the vasculature. FIG. 1 shows a common carotid artery (CCA) 10, which bifurcates into the external carotid artery (ECA) 12 and the internal carotid artery (ICA) 14. A lesion 16 is schematically shown within the ICA 14, just above a bifurcation point 17. An arterial sheath may be advanced up through the vasculature to a point within the CCA 10 and an inflatable occlusion balloon carried by the arterial sheath may be used to occlude anterograde blood flow through the CCA 10. The CCA 10 may be reached by advancing through the arterial system to the CCA 10. The arterial system may be accessed via a number of different arteries, but in some instances, the arterial system may be accessed via one of the patient's femoral arteries, as the patient has a femoral artery extending through the groin and down either leg. In some instances, other arteries providing a shorter path to the CCA 10 may be utilized.

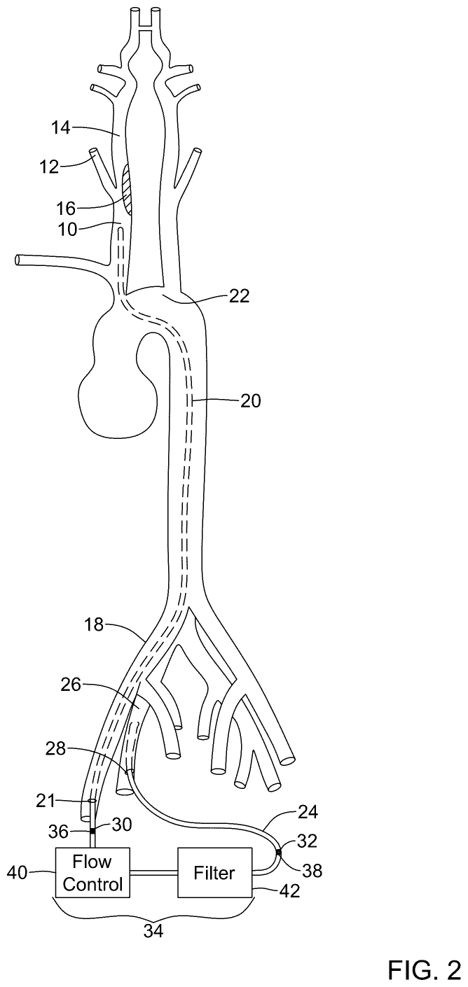

FIG. 2 is a schematic view of a portion of the patient's vasculature providing an illustrative path for advancing an arterial sheath from a femoral artery 18. An arterial sheath 20 is schematically seen, passing from an access point 21 within the femoral artery 18, through an aortic arch 22 and into the common carotid artery 10. A Seldinger technique may be used to create the access point 21 under fluoroscopic guidance. In some instances, a Seldinger technique involves introducing a needle into the vasculature, followed by advancing a wire through the needle and into the vein before the needle is withdrawn. The arterial sheath 20, along with an introducer, may be advanced over the wire and into the femoral artery 18. The arterial sheath 20 may subsequently be advanced through the vasculature to reach the common carotid artery 10, for example.

In some instances, either before or after the arterial sheath 20 has been introduced into the femoral artery 18, a venous sheath 24 may be introduced into the venous system. In some instances, this involves a femoral vein 26, although other access points to the venous system are contemplated. The venous sheath 24 may be introduced into the femoral vein 26 at an access point 28 in a manner similar to that used for introducing the arterial sheath 20 into the femoral artery 18. As an example, a Seldinger technique may be used under fluoroscopic guidance.

A proximal end 30 of the arterial sheath 20 and a proximal end 32 of the venous sheath 24 may be joined to a fluid path 34. In some instances, the fluid path 34 may simply represent one or more fittings or connections that allow the proximal end 30 of the arterial sheath 20 and the proximal end 32 of the venous sheath 24 to be fluidly coupled together. In some instances, the proximal end 30 of the arterial sheath 20 may include a fitting 36 and the proximal end 32 of the venous sheath 24 may include a fitting 38 that permits a direct connection between the proximal end 30 of the arterial sheath 20 and the proximal end 32 of the venous sheath 24.

In some instances, once the fluid path 34 between the arterial sheath 20 and the venous sheath 24 has been established, the arterial sheath 20 may be advanced further into the femoral artery 18 (or other artery if used) towards the CCA 10. In some instances, the fitting 36 and the fitting 38 may each be adapted to be coupled with one or more additional components within the fluid path 34. As an example, in some instances the fluid path 34 may include a flow control device 40. The flow control device 40 may include an on/off valve that may be adjusted by an operator to either permit retrograde blood flow through the fluid path 34, or to prevent retrograde blood flow through the fluid path 34. In some instances, the flow control device 40 may be adapted to be able to adjust the relative retrograde blood flow through the fluid path 34, for example. In some instances, the fluid path 34 may include a filter 42. The filter 42 may be adapted to screen out any particles over a threshold diameter, for example. In some instances, the filter 42 may be adapted to screen out some particles, while the venous system itself will screen out additional particles.

While the flow control device 40 is shown coupled directly to the arterial sheath 20 while the filter 42 is shown coupled directly to the venous sheath 24, it will be appreciated that this is merely illustrative, as the flow control device 40 and the filter 42 may be connected in any desired order. In some instances, the fluid path 34 may include the flow control device 40 but may not include the filter 42. In some instances, the fluid path 34 may include the filter 42 but may not include the flow control device 40.

In some instances, a retrograde blood flow is achieved through the arterial sheath 20 as a result of the arterial sheath 20 being fluidly coupled to the relatively high pressure of the arterial system while the vascular sheath 24 is fluidly coupled to the relatively low pressure of the venous system. In some instances, the retrograde blood flow resulting from these pressure differences means that any debris that may be knocked loose or otherwise dislodged while advancing the arterial sheath 20 through the vasculature will be carried through the fluid path 34 into the venous system. In some instances, at least some of the debris may be captured by the filter 42. The filter 42 may also capture additional debris that may be dislodged while performing various processes such as stenting the lesion 16.

Providing retrograde flow through the arterial sheath 20 allows for any debris that is created or knocked loose during any procedure within the CCA 10, the ECA 12 and/or the ICA 14 to be carried away from the CCA 10, the ECA 12 and/or the ICA 14. Some patients may be sensitive to having limited or no blood flow through the ECA 12 and/or the ICA 14 as a result of establishing retrograde flow through the arterial sheath 20. In some instances, patients having poor blood flow to the ipsilateral vessels from contralateral and/or vertebral vessels may suffer from cerebral hypoperfusion and discomfort. As an example, patients may suffer complications such as temporary facial paralysis as a result. Retrograde blood flow through the arterial 20 may be provided while also providing for limited blood flow into either the ECA 12 or the ICA 14. The vasculature within a patient's head includes several connections between the ECA 12 and the ICA 14, meaning that antegrade blood flow provided within the ECA 12 is able to reach vessels supplied by the ICA 14, and antegrade blood flow provided within the ICA 14 is able to reach vessels supplied by the ECA 12.

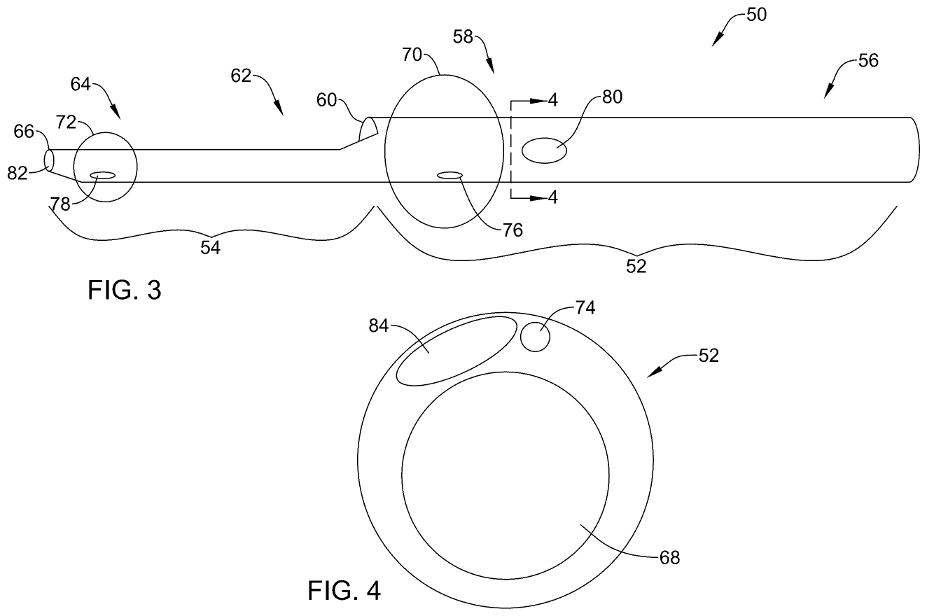

FIG. 3 is a schematic view of an illustrative catheter 50 and FIG. 4 is a cross-sectional view taken along the line 4-4 of FIG. 3. The illustrative catheter 50 may be considered as being an example of the arterial sheath 20. As such, the catheter 50 may be considered as being a reverse flow catheter for performing procedures such as carotid artery stenting. The catheter 50 may be considered as including a proximal catheter segment 52 and a distal catheter segment 54. The proximal catheter segment 52 extends from a proximal region 56 to a distal region 58, the distal region 58 extending distally to a distal end 60. The proximal catheter segment 52 is adapted to be positioned within the vasculature such that the proximal catheter segment 52 terminates with its distal end 60 positioned within the CCA 10. In some instances, the proximal catheter segment 52 may have an 8 French outer diameter. The distal catheter segment 54 extends from a proximal region 62 to a distal region 64, the distal region 64 extending distally to a distal end 66. The distal catheter segment 54 is adapted to be positioned within the vasculature such that the distal catheter segment 54 terminates with its distal end 66 within the ECA 12.

The catheter 50 includes a working lumen 68 that extends through the proximal catheter segment 52 and terminates at the distal end 60 of the proximal catheter segment 52. In some instances, the working lumen 68 may be adapted to accommodate retrograde blood flow through the working lumen 68. The working lumen 68 may be adapted to accommodate other devices such as stent delivery catheters being advanced through the working lumen 68. In some instances, the working lumen 68 may be adapted to accommodate retrograde blood flow through the working lumen 68 at the same time that another device, such as a stent delivery catheter, is being advanced through the working lumen 68. As an example, the working lumen 68 may have a 6 French inner diameter.

The catheter 50 includes a CCA occlusion balloon 70 that is secured within the distal region 58 of the proximal catheter segment 52. The CCA occlusion balloon 70 may be adapted to occlude the CCA 10 when the catheter 50 is properly positioned relative to the CCA 10. In some instances, the CCA occlusion balloon 70 may have an inflated diameter that is sufficient to occlude the CCA 10. The catheter 50 includes an ECA occlusion balloon 72 that is secured within the distal region 64 of the distal catheter segment 54. The ECA occlusion balloon 72 may be adapted to occlude the ECA 12 when the catheter 50 is properly positioned relative to the CCA 10. In some instances, the ECA occlusion balloon 72 may have an inflated diameter that is sufficient to occlude the ECA 12.

In some instances, the catheter 50 includes an inflation lumen 74 that extends to and is fluidly coupled with the CCA occlusion balloon 70 such that an inflation fluid such as saline may be provided to the CCA occlusion balloon 70 in order to inflate the CCA occlusion balloon 70. In some instances, the inflation lumen 74 may also extend to and is fluidly coupled with the ECA occlusion balloon 72 such that an inflation fluid such as saline may be provided to the ECA occlusion balloon 72 in order to inflate the ECA occlusion balloon 72. In some instances, a second inflation lumen (not shown) may extend to and be fluidly coupled with the ECA occlusion balloon 72. In some instances, the catheter 50 includes a proximal inflation port 74 that is fluidly coupled with an interior of the OCC occlusion balloon 70 as well as being fluidly coupled with the inflation lumen 74. The catheter 50 may include a distal inflation port 76 that is fluidly coupled with an interior of the ECA occlusion balloon 72 as well as being fluidly coupled with either the inflation lumen 74 or another lumen (not shown).

In some instances, the catheter 50 includes a proximal shunt port 80 that is disposed within the proximal catheter segment 52 proximal of the CCA occlusion balloon 70 and a distal shunt port 82 that is disposed within the distal catheter segment 54 distal of the ECA occlusion balloon 72. In some instances, the distal shunt port 82 is disposed at the distal end 66 of the distal catheter segment 54. A shunt lumen 84 extends between the proximal shunt port 82 and the distal shunt port 84. This allows antegrade blood flow from proximal of the CCA occlusion balloon 70 to enter the proximal shunt port 80, pass through the shunt lumen 84, and exit through the distal shunt port 82. As a result, even though the overall blood flow through the ICA 14 and the CCA 10 is retrograde, a small amount of antegrade blood flow is allowed to pass into the ECA 12. As noted, due to vascular connections between blood vessels supplied by the ECA 12 and blood vessels supplied by the ICA 14, this means that sufficient blood is provided to both the ECA 12 and the ICA 14 to avoid deleterious effects that may otherwise occur as a result of the retrograde blood flow. Given the relative positions of the proximal shunt port 80 and the distal shunt port 82, the shunt lumen 84 may be seen as extending through part of the proximal catheter segment 52 and extending through all of the distal catheter segment 54. In some instances, the shunt lumen 84 may have an ovoid-shaped cross-sectional profile.

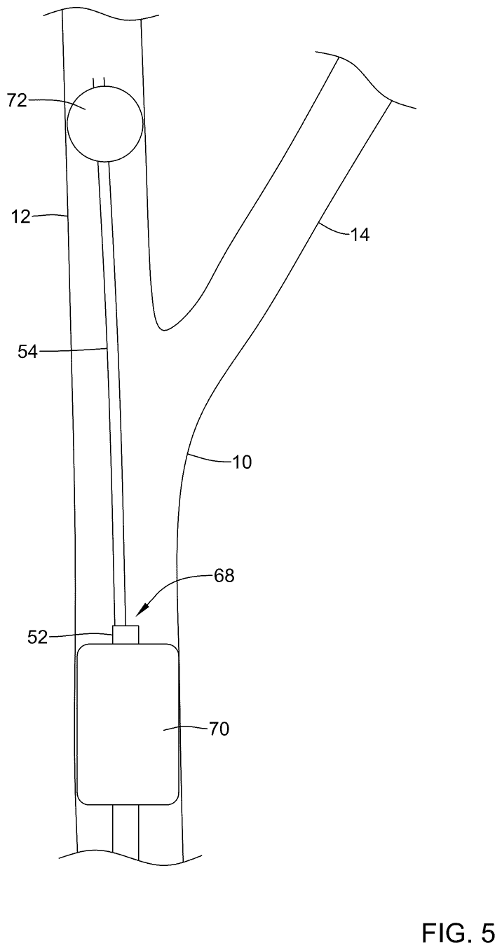

FIGS. 5 through 7 are schematic views providing an example of using the catheter 50. It will be appreciated that FIGS. 5 through 7 are schematic in nature and may not display all features and elements of the catheter 50. In FIG. 5, the catheter 50 has been advanced through the vasculature to a point in which the proximal catheter segment 52 and the CCA occlusion balloon 70 is positioned in the CCA 10 and the distal catheter segment 54 and the ECA occlusion balloon 72 is positioned in the ECA 12. As shown, the CCA occlusion balloon 70 has been inflated to occlude the CCA 10, thereby causing retrograde blood flow to flow down the ICA 14 into the CCA 10 and into and through the working lumen 68 formed within the proximal catheter segment 52. In order to achieve retrograde flow, it will be appreciated that the working lumen 68 may be placed into fluid communication with the patient's venous system, such as via the venous sheath 24 shown in FIG. 2. In some instances, the working lumen 68 may simply drain into an external container. The ECA occlusion balloon 72 has also been inflated, thereby occluding antegrade blood flow within the ECA 12, apart from blood flowing through the shunt lumen 84.

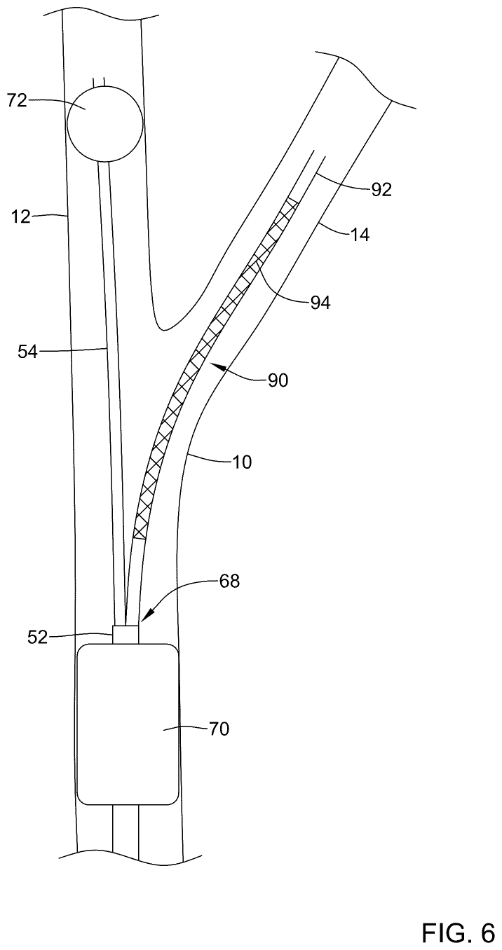

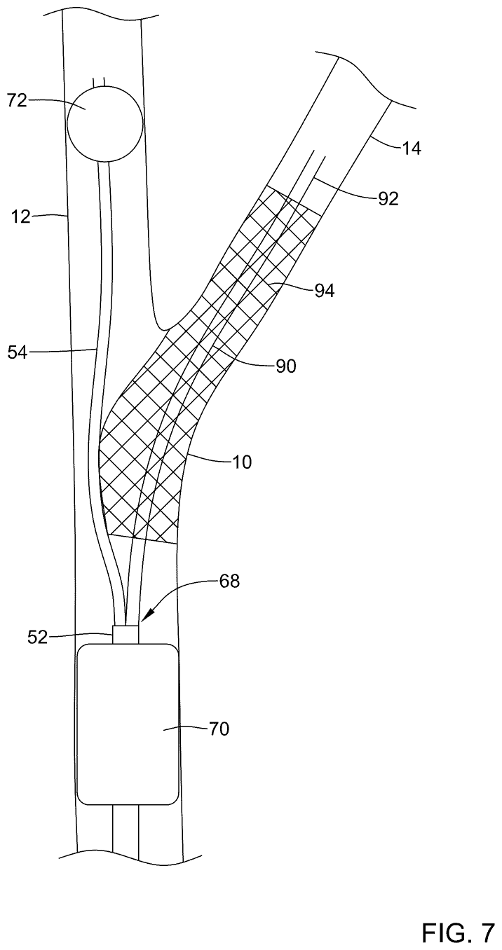

Moving to FIG. 6, it can be seen that a second catheter 90 has been advanced through the working lumen 68 of the proximal catheter segment 52. In some instances, the second catheter 90 may be advanced over a guidewire (not shown). The second catheter 90 has been advanced into the ICA 14. In some instances, the second catheter 90 includes an elongate shaft 92 bearing an expandable stent 94. In some instances, the expandable stent 94 may be self-expanding, and may expand when a delivery sheath (not shown) is withdrawn proximally, allowing the expandable stent 94 to expand. This may be seen in FIG. 7, where the expandable stent 94 has been allowed to expand. As noted, the expandable stent 94 may expand as a result of removal of a previously constraining sheath. In some instances, the expandable stent 94 may be balloon-expandable, meaning that a balloon (not shown) formed as part of the elongate shaft 92 may be temporarily inflated (and subsequently deflated) in order to expand the expandable stent 94. In some instances, expanding the expandable stent 94 (or allowing the expandable stent 94 to expand) may push the distal catheter segment 54 to the side (as shown in FIG. 7).

Once the expandable stent 94 has been expanded, the second catheter 90 may be withdrawn proximally and removed from the patient. The CCA occlusion balloon 70 may be deflated in order to no longer occlude the CCA 10, and the ECA occlusion balloon 72 may be deflated in order to no longer occlude the ECA 12. The catheter 50 may then be withdrawn from the patient. At some point during the process, the retrograde blood flow caused by a fluid connection between the working lumen 68 and the venous sheath 24 (FIG. 2) may be disconnected, thereby allowing normal blood flow to resume. In some instances, the fluid connection between the working lumen 68 and the venous sheath 24 may be disconnected last, in order to ensure that any debris caused by catheter movement is removed via the retrograde flow.

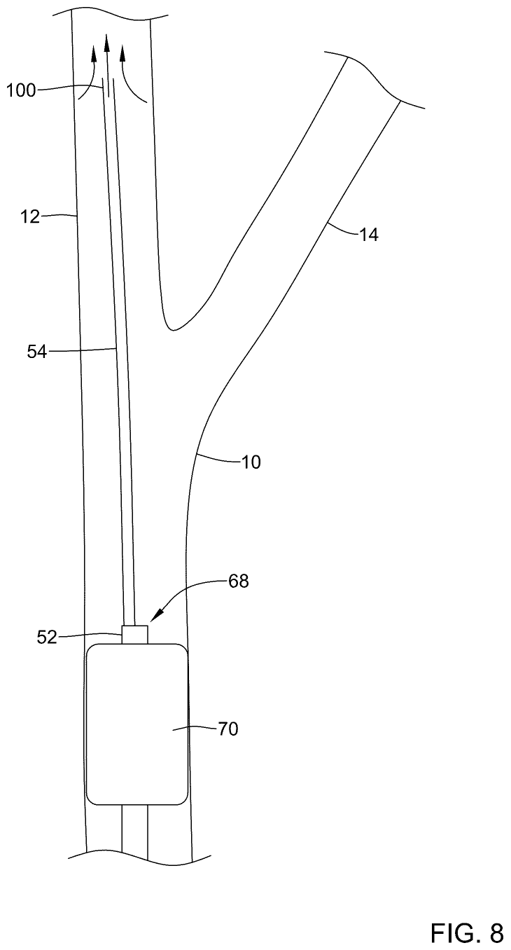

FIG. 8 provides a schematic view of an alternate to the catheter 50. Rather than including the ECA occlusion balloon 72, the distal catheter segment 54 may instead include a jet 100 from which a high velocity stream of blood that passed through the blood shunt lumen 84 (FIG. 4) exits. In some instances, the velocity of the blood exiting the jet 100 may be increased by decreasing a diameter of the jet 100. By having blood exit the jet 100 at a sufficient velocity, blood flow is caused to flow in an antegrade direction. In some instances, this may provide sufficient antegrade blood flow without occluding the ECA 12.

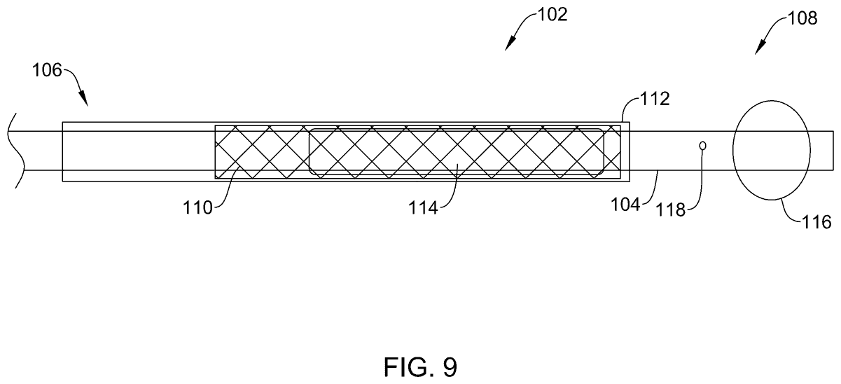

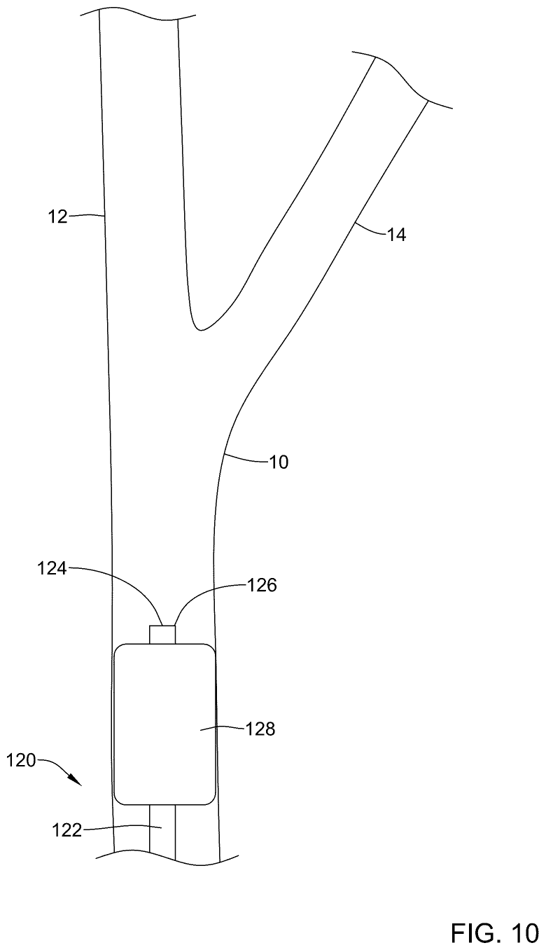

In FIGS. 5 through 7, the catheter 50 is used to occlude the CCA 10 and the ECA 12, and to accommodate a second catheter (the second catheter 90) through the working channel 68 of the catheter 50 in order to deploy the expandable stent 92 within the ICA 14. The catheter 50 is also used as the arterial sheath 20, forming a retrograde blood flow path in combination with the venous sheath 24 (FIG. 2). In some instances, rather than creating retrograde blood flow through the ICA 14, and occluding blood flow within the ECA 12, it may be desirable to instead occlude blood flow within the ICA 14 and to allow retrograde blood flow through the ECA 12. FIG. 9 is a schematic view of an illustrative catheter 102 that may be used to occlude the ICA 14 and deliver an expandable stent to the ICA 14 while another catheter is used to occlude the CCA 10 and create the retrograde blood flow path in combination with the venous sheath 24.

In FIG. 9, the catheter 102 includes an elongate shaft 104 extending from a proximal region 106 to a distal region 108. An expandable stent 110 is disposed on the elongate shaft 104 within the distal region 108, and is held in its collapsed configuration by being constrained by a stent deployment sheath 112. Subsequent removal of the stent deployment sheath 112 will allow the expandable stent 110 to expand to its expanded configuration, as the expandable stent 110 may be considered as being a self-expanding stent. The catheter 102 includes a post-stenting dilation balloon 114 that may be used to more fully expand the expandable stent 110, and/or to help compress a lesion (such as the lesion 16 shown in FIG. 1). The catheter 102 includes an ICA occlusion balloon 116 that may be inflated in order to occlude the ICA 14, or deflated to not occlude the ICA 14. The catheter 102 also includes a flushing port 118 that may be used subsequent to stent deployment to flush saline through the ICA 14 to help flush out any possible debris prior to deflating the ICA occlusion balloon 116. While a single flushing port 118 is shown, in some instances there may be two, three or more flushing ports 118 arranged circumferentially around the elongate shaft 104, for example.

While not expressly shown, it will be appreciated that the elongate shaft 104 may include several internal lumens, including a blood shunt path that allows some antegrade blood flow through the ICA 14. The elongate shaft 104 may include a flushing lumen for providing saline or other suitable flushing fluid to the flushing port 118. The elongate shaft 104 may include an inflation lumen for inflating and deflating the ICA occlusion balloon 116. The elongate shaft 104 may include an inflation lumen for inflating and deflating the post-stenting dilation balloon 114.

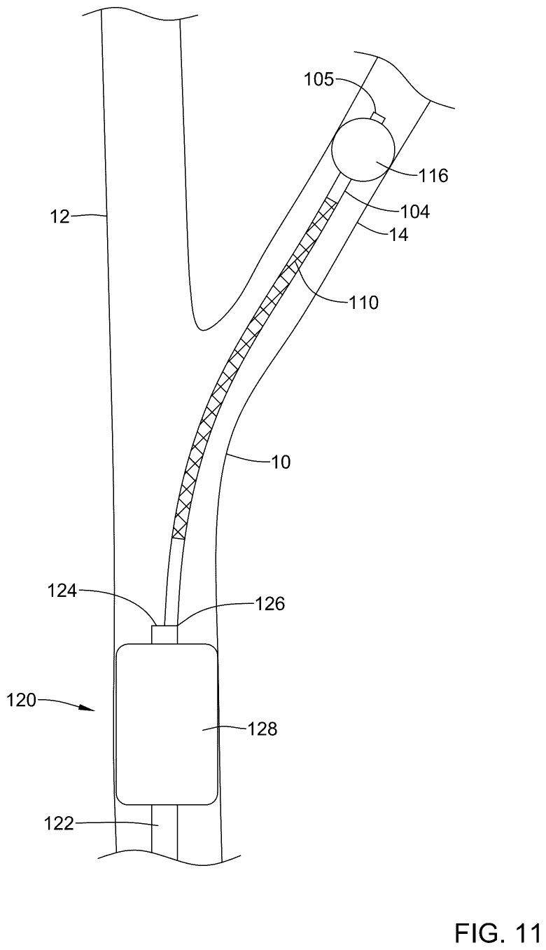

As noted, the catheter 102 may be used in combination with another catheter. FIG. 10 shows a catheter 120 shown disposed within the CCA 10. The catheter 120 may be considered as being an example of the arterial sheath 20 (FIG. 2), and thus may extend proximally to a fluid coupling with the venous sheath 24. The catheter 120 includes an elongate shaft 122 that terminates at an opening 124 at a distal end 126 of the elongate shaft 122. The opening 124 is adapted to permit retrograde blood flow into and through the elongate shaft 122. The catheter 120 includes a CCA occlusion balloon 128 that may be inflated in order to occlude the CCA 10. In some instances, the CCA occlusion balloon 128 may be a compliant silicone balloon, for example.

In FIG. 11, the catheter 102 has been advanced through the CCA occlusion balloon 128 of the catheter 120. In some instances, as shown in FIG. 11, the catheter 102 may be advanced through the elongate shaft 122 of the catheter 120. As shown, the catheter 102 has been advanced into the ICA 14, and the ICA 14 occlusion balloon 116 has been inflated, thereby occluding the ICA 14. There is some antegrade blood flow through the ICA 14, via the blood exiting a distal end 105 of the elongate shaft 104. As noted, this represents a blood flow shunt from a position proximal of the CCA occlusion balloon 128 (of the catheter 120), through the elongate shaft 114 and out the distal end 105 of the elongate shaft 104. By providing some blood flow through the ICA 14, and by virtue of the shared connections in the head between blood vessels supplied by the ECA 12 and blood vessels supplied by the ICA 14, adequate blood flow should be available to prevent possible side effects in patients who are otherwise susceptible to possible side effects as a result of occluding blood flow through either the ECA 12 or the ICA 14.

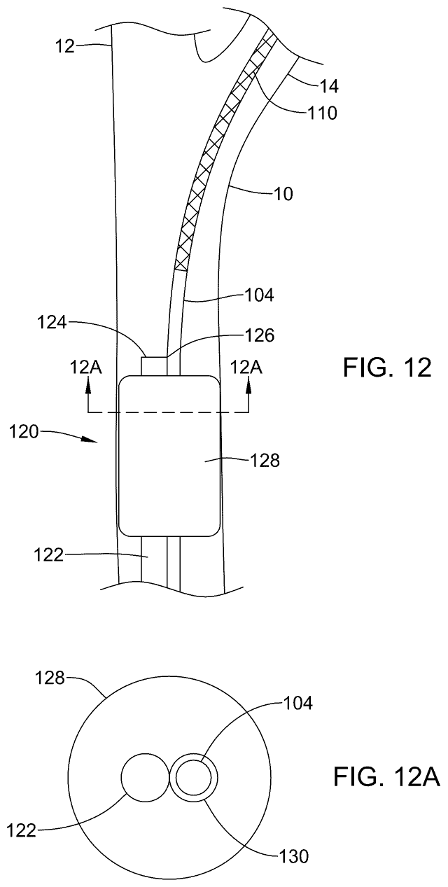

In some instances, rather than passing the catheter 102 through the elongate shaft 122 of the catheter 120, the catheter 102 may pass through the OCC occlusion balloon 128 adjacent the elongate shaft 122, rather than coaxially with the elongate shaft 122. FIG. 12 shows the catheter 102 passing through the OCC occlusion balloon 128 adjacent the elongate shaft 122 of the catheter 120. FIG. 12A is a cross-sectional view taken along the line 12A-12A of FIG. 12. The elongate shaft 122 may be seen as extending through the OCC occlusion balloon 128. Adjacent the elongate shaft 122 of the catheter 120 is an aperture 130 that extends axially through the OCC occlusion balloon 128 and is adapted to accommodate the elongate shaft 104 of the catheter 102 therethrough. When the OCC occlusion balloon 128 is inflated, the aperture 130 pushes against the side of the elongate shaft 104 and seals against the side of the elongate shaft 104.

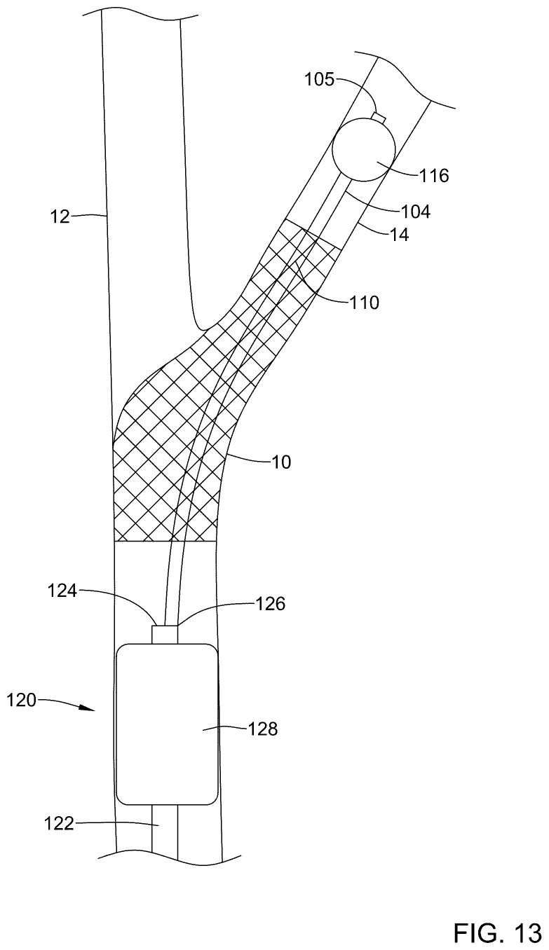

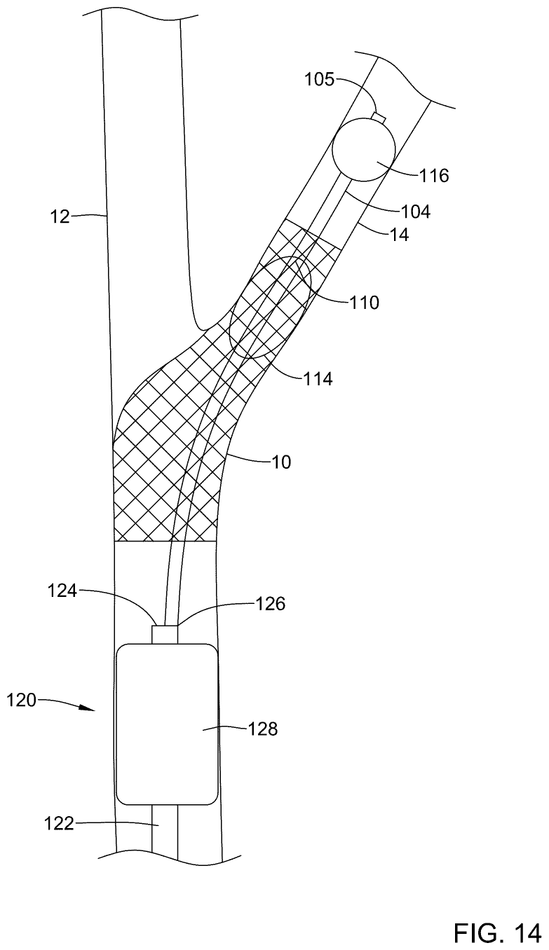

In FIG. 13, the expandable stent 110 can be seen as having been deployed. This may occur by withdrawing proximally the stent deployment sheath 112, for example. The stent deployment sheath 112 has been removed and thus is not visible in this view. Subsequent to deploying the expandable stent 110, the post-stenting dilation balloon 114 may be inflated, as seen in FIG. 14. The post-stenting dilation balloon 114 may be used to further expand the expandable stent 110, for example, or to help enlarge an opening within the lesion 16 (FIG. 1) formed by the stent 110. In some instances, after the expandable stent 110 has been deployed, and the post-stenting dilation balloon 114 has been used, there may be a desire to flush any possible debris out of the ICA 14 while the ICA 14 is still occluded.

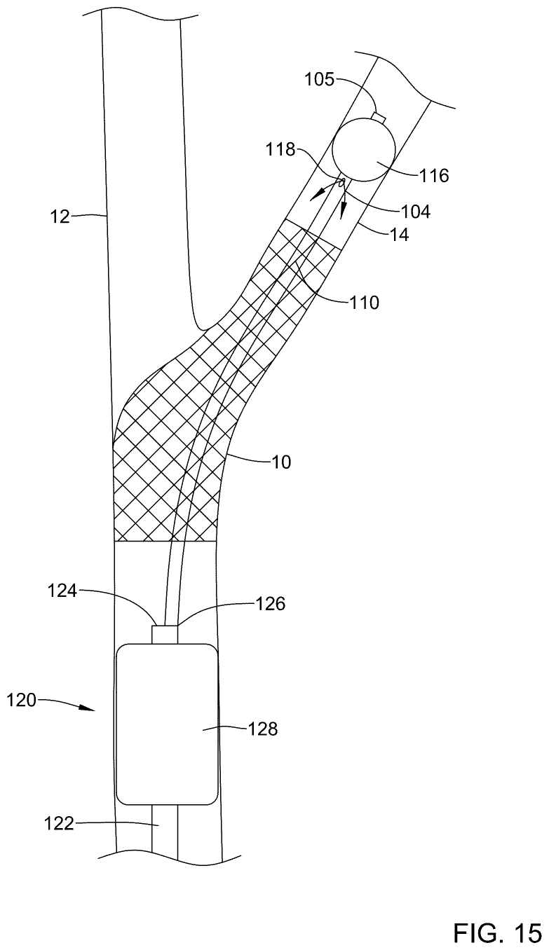

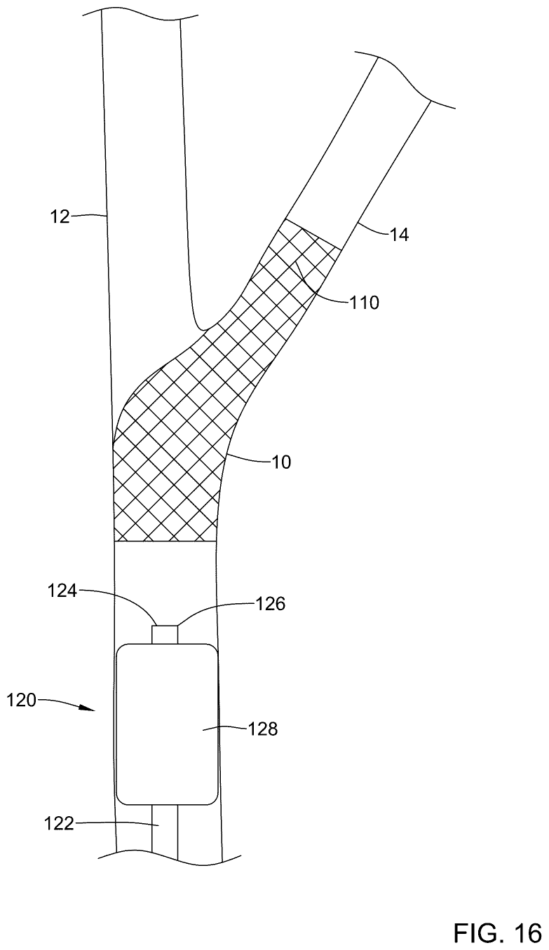

In FIG. 15, saline is being provided to the flushing port(s) 118 such that any debris such as embolic particles are flushed down the ICA 14 and into the catheter 120 in the retrograde blood flow path. Once the ICA 14 has been flushed with saline, the catheter 102 may be removed. This may be seen in FIG. 16, which shows the expandable stent 110 deployed through where the ICA 14 bifurcates from the CCA 10. Subsequently, the CCA occlusion balloon 128 may be deflated and the catheter 120 may be withdrawn as well. At some point during the process, the retrograde blood flow caused by a fluid connection between the elongate shaft 122 and the venous sheath 24 (FIG. 2) may be disconnected, thereby allowing normal blood flow to resume.

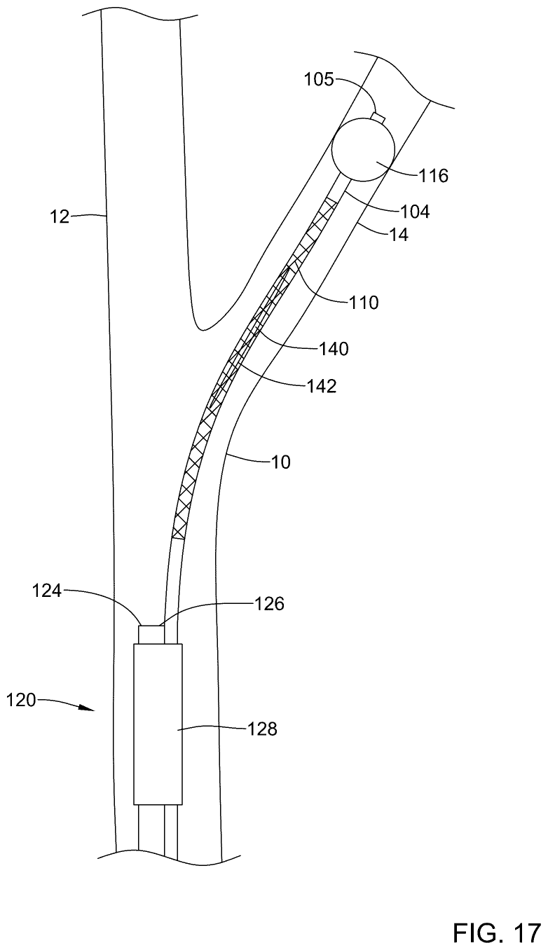

FIG. 17 provides a schematic view of an alternate to the catheter 102. In this view, the catheter 102 includes a blood shunt opening 140 that is covered by a filter 142. The blood shunt opening 140 may allow filtered blood to enter into the blood shunt opening 140 when the CCA occlusion balloon 128 is deflated (as shown) and subsequently exit through the distal end 105 of the elongate shaft 104.

The materials that can be used for the various components of the medical devices described herein may include those commonly associated with medical devices. The medical devices described herein, as well as individual components thereof, be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical devices described herein may also be doped with, made of, or otherwise include a radiopaque material. Radiopaque materials are understood to be materials capable of producing a relatively bright image on a fluoroscopy screen or another imaging technique during a medical procedure. This relatively bright image aids in determining a location of a medical device that includes a radiopaque material. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into various medical devices to achieve the same result.

The medical devices described herein, as well as portions and components thereof, may be made of the same material along its length, or in some embodiments, can include portions or sections made of different materials. In some embodiments, materials may be chosen to impart varying flexibility and stiffness characteristics to different portions. For example, different portions of a component, such as a proximal section and a distal section, may be formed of different materials, for example, materials having different moduli of elasticity, resulting in a difference in flexibility. In some embodiments, the material used to construct a proximal section may be relatively stiff for pushability and torqueability, and the material used to construct a distal section may be relatively flexible by comparison for better lateral trackability and steerability. For example, a proximal section may be formed of straightened 304v stainless steel wire or ribbon and a distal section may be formed of a straightened super elastic or linear elastic alloy, for example a nickel-titanium alloy wire or ribbon.

In embodiments where different portions of the medical devices described herein are made of different materials, the different portions can be connected using a suitable connecting technique and/or with a connector. For example, the different portions may be connected using welding (including laser welding), soldering, brazing, adhesive, or the like, or combinations thereof. These techniques can be utilized regardless of whether or not a connector is utilized. An example of a connector is a structure such as a hypotube or a coiled wire which has an inside diameter sized appropriately to receive and connect to the ends of the proximal portion and the distal portion.

A sheath or covering (not shown) may be disposed over portions or all of the medical devices described herein. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the medical devices described herein may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the medical devices described herein. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Having thus described several illustrative embodiments of the present disclosure, those of skill in the art will readily appreciate that yet other embodiments may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A reverse flow catheter for performing carotid artery stenting, the reverse flow catheter comprising:
a proximal catheter segment extending from a proximal region to a distal region including a distal end, the proximal catheter segment adapted to terminate with its distal end within a common carotid artery (CCA);
a distal catheter segment extending from a proximal end to a distal region including a distal end, the distal catheter segment extending distally from the distal end of the proximal catheter segment, the distal catheter segment adapted to terminate within an external carotid artery (ECA);
a working lumen extending through the proximal catheter segment and terminating at the distal end of the proximal catheter segment;
a CCA occlusion balloon secured within the distal region of the proximal catheter segment;
an ECA occlusion balloon secured within the distal region of the distal catheter segment;
a first shunt port disposed within the proximal catheter segment proximal of the CCA occlusion balloon;
a second shunt port disposed within the distal catheter segment distal of the ECA occlusion balloon; and
a shunt lumen extending between the first shunt port and the second shunt port in order to allow antegrade blood flow from proximal of the CCA occlusion balloon to distal of the ECA occlusion balloon.

2. The reverse flow catheter of claim 1, wherein the shunt lumen extends through part of the proximal catheter segment and through the distal catheter extension.

3. The reverse flow catheter of claim 2, wherein the shunt lumen comprises an ovoid-shaped shunt lumen.

4. The reverse flow catheter of claim 1, further comprising an inflation lumen extending to and fluidly coupled with the CCA occlusion balloon.

5. The reverse flow catheter of claim 4, wherein the inflation lumen also extends to and is fluidly coupled with the ECA occlusion balloon.

6. The reverse flow catheter of claim 1, wherein the working lumen is adapted to accommodate retrograde blood flow therethrough.

7. The reverse flow catheter of claim 1, wherein the catheter body has an 8 French outer diameter.

8. The reverse flow catheter of claim 7, wherein the working lumen has a 6 French inner diameter.

9. The reverse flow catheter of claim 1, wherein the working lumen is adapted to accommodate a stent delivery catheter extended through the working lumen and into the interior carotid artery (ICA).

10. A catheter, comprising:
a proximal catheter segment extending from a proximal region to a distal region including a distal end, the proximal catheter segment adapted to terminate with its distal end within a common carotid artery (CCA), the proximal catheter segment having an 8 French outer diameter;
a distal catheter segment extending from a proximal end to a region including a distal end, the distal catheter segment extending distally from the distal end of the catheter body, the distal catheter segment adapted to terminate within an external carotid artery (ECA);
a working lumen extending through the proximal catheter segment and terminating at the distal end of the proximal catheter segment, the working lumen having a 6 French inner diameter;
a CCA occlusion element secured within the distal region of the catheter body;
an ECA occlusion element secured within the distal region of the distal catheter segment; and
a blood shunt path extending from a position proximal of the CCA occlusion element to a position distal of the ECA occlusion element in order to allow antegrade blood flow from proximal of the inflatable CCA occlusion balloon to distal of the inflatable ECA occlusion balloon.

11. The catheter of claim 10, further comprising:
a proximal blood shunt port fluidly coupled with a proximal end of the blood shunt path; and
a distal blood shunt port fluidly coupled with a distal end of the blood shunt path.

12. The catheter of claim 10, wherein the working lumen is adapted to accommodate retrograde blood flow through the working lumen simultaneously with a stent delivery catheter being advanced through the working lumen.

13. A reverse flow medical device combination for performing carotid artery stenting, the reverse flow medical device combination comprising:
a first catheter including:
a catheter body extending from a proximal region to a distal region including a distal end, the catheter body adapted to terminate with its distal end within a common carotid artery (CCA);
a working lumen extending through the catheter body and terminating at the distal end of the catheter body;
a first inflatable balloon secured within the distal region of the catheter body of the first catheter and adapted when inflated to occlude the CCA; and
a second catheter including:
a catheter body extending from a proximal region to a distal region including a distal end, the catheter body adapted to terminate with its distal end within an internal carotid artery (ICA);
an expandable stent disposed over the catheter body;
a second inflatable balloon secured within the distal region of the catheter body of the second catheter;
a blood lumen extending through the catheter body to a position distal of the second inflatable balloon in order to allow antegrade blood flow from a position proximal of the first inflatable balloon to a position distal of the second inflatable balloon.

14. The reverse flow medical device combination of claim 13, wherein the second catheter further comprises a stent deployment sheath that is adapted to be withdrawn proximally in order to deploy the stent.

15. The reverse flow medical device combination of claim 13, wherein the second catheter further comprises a post-stenting dilation balloon.

16. The reverse flow medical device combination of claim 13, wherein the second catheter further comprises one or more flushing ports disposed proximal of the second inflatable balloon.

17. The reverse flow medical device combination of claim 13, wherein the second catheter is adapted to extend coaxially with the first catheter.

18. The reverse flow medical device combination of claim 17, wherein the second catheter is adapted to extend within the working lumen of the first catheter.

19. The reverse flow medical device combination of claim 13, wherein the second catheter is adapted to extend next to the first catheter.

20. The reverse flow medical device combination of claim 19, wherein the first inflatable balloon comprises a lumen extending therethrough adapted to accommodate the first catheter within the lumen.
